(19) United States
(12) Patent Application Publication
(10) Pub. No.: US 2021/0307645 A1
(43) Pub. Date: Oct. 7, 2021

(54) SPIROMETER APPARATUS

(71) Applicant: SMITHS MEDICAL INTERNATIONAL LIMITED, Ashford (GB)

(72) Inventor: Mohammad Qassim Mohammad Khasawneh, Canterbury (GB)

(73) Assignee: SMITHS MEDICAL INTERNATIONAL LIMITED, Ashford (GB)

(21) Appl. No.: 17/057,198

(22) PCT Filed: Jun. 3, 2019
(86) PCT No.: PCT/GB2019/000080

§ 371 Date: Nov. 20, 2020

(30) Foreign Application Priority Data
Jun. 9, 2018 (GB) 1809558.8

(51) Int. Cl. A61B 5/087; A61B 5/00; A61B 5/15

(52) U.S. Cl. CPC A61B 5/0871; A61B 5/6898; A61B 2562/0247; A61B 5/150809; A61B 5/150824; A61B 5/0022

(57) ABSTRACT

An incentive spirometer has a housing (1) with a vertical cylinder (4) connected at its upper end to one end of a gas passage (8) that is connected at its opposite end to an inlet tube (10) and mouthpiece (11). A piston (20) in the cylinder is moved up when the patient inhales through the mouthpiece and thereby creates a reduced pressure at the upper end of the cylinder. The spirometer also includes a flow sensor (30) located in the gas passage (8) that generates a wireless signal indicative of gas flow along the passage. A monitor (40), such as a suitably programmed mobile phone, is located separately of the spirometer housing (1) and responds to the output of the sensor (30). The monitor (40) records and provides feedback to the user indicative of his use of the apparatus.

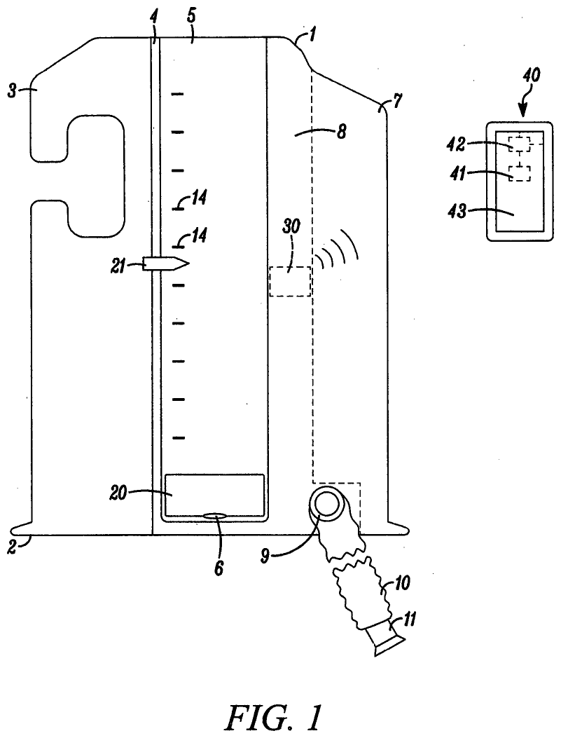

This invention relates to incentive spirometer apparatus of the kind having a housing, a cylinder, a patient inhalation inlet and a gas passage between the inhalation inlet and one end of the cylinder, the cylinder containing a piston movable along the cylinder against a restoring force, such that an inhalation breath applied to the inlet causes a reduced pressure at one end of the cylinder so as to displace the piston against the restoring force.

Incentive spirometers are used to help patients improve lung function, such as following surgery, prolonged anaesthesia or following a chest injury or disease. They are also used by wind instrument players and sports people to improve lung capacity.

Incentive spirometers usually have a vertical cylinder containing a movable piston slidable along the cylinder. A flexible tube is terminated at one end by a mouthpiece and has its other end connected to an opening in the spirometer that in turn connects with the upper end of the cylinder. When the patient inhales through the mouthpiece it creates a reduced pressure in the tube that is communicated to the upper end of the cylinder. This causes the piston to rise up the cylinder. The cylinder is transparent, or has a transparent window, and is graduated by volume up its height so that the user can compare the position of the piston against the graduated scale. The aim of the patient is to inhale slowly and deeply to draw the piston up to a target volume and to maintain this position for as long as possible.

It has been proposed to incorporate in the spirometer some means to record its use. U.S. Pat. No. 6,238,353 describes a spirometer with a goal recording counter that can be slid up or down the outside of the cylinder to the target volume. The counter includes an optical sensor that detects when the piston is aligned with the counter, that is, when the piston has been raised to its target volume. The piston is detected by infra-red radiation transmitted from the counter through the wall of the cylinder and reflected from the piston. The counter maintains a count of the number of times the piston has been raised to its target volume so that the user can determine when he has correctly completed his therapy session. US20180000379 describes a similar spirometer with a sensor that generates an alarm signal until the piston has been raised to its target volume.

These previous arrangements rely on detecting when the piston reaches a target position along the cylinder. One problem with these arrangements is that the sensor may obscure the cylinder in the target region of interest, making it more difficult for the user to determine whether or not he has achieved the correct inhalation goal. Another problem is that such arrangements do not monitor use that fails to meet the target level. No account is taken of an inhalation breath that raises the piston close to but below the target and no measure can be given of the extent to which the target use has not been met. This can be very disheartening for the patient who may be carrying out the inhalation therapy diligently and improving progress but the monitor does not reflect this.

It is an object of the present invention to provide alternative spirometer apparatus.

According to the present invention there is provided inhalation spirometer apparatus of the above-specified kind, characterised in that the spirometer apparatus also includes an electronic pressure or flow sensor responsive to change in pressure or flow of gas within the apparatus during use and a monitor for receiving an output signal from the sensor and for processing and utilising the output signal.

The sensor is preferably arranged to provide a wireless output to the monitor, the monitor being located externally of the spirometer housing. The housing is preferably arranged to stand with the cylinder extending vertically, the restoring force being provided by the effect of gravity on the mass of the piston. The apparatus may include a flexible tube having one end connected with the inhalation inlet and with a mouthpiece at its opposite end. The sensor is preferably located in the gas passage between the inhalation inlet and the one end of the cylinder. The monitor may be provided by a mobile phone arranged to receive wireless signals from the sensor, the mobile phone being programmed to provide an indication to the user of feedback regarding use of the apparatus. The apparatus may include a slider that can be manually positioned by the user along the cylinder to indicate a target volume.

Spirometer apparatus according to the present invention will now be described, by way of example, with reference to the accompanying drawing, which is a front elevation view.

The spirometer has a moulded plastics housing 1 with a flat base 2 on which the spirometer stands during use. The housing 1 has a carrying handle 3 towards its left-hand upper end and a vertical cylinder 4 of circular section positioned midway across and extending up the entire height of the spirometer. The cylinder 4 is either entirely transparent or has a transparent window extending along its height on the front surface 5 facing the user. The cylinder 4 is sealed externally apart from a small vent aperture 6 at its lowest point, the purpose of which will become apparent later. To the right of the cylinder 4 extends a flat, hollow, vertical wall 7 having a narrow vertical air channel or gas passage 8 within it that opens at its upper end into the upper end of the cylinder 4 and at its lower end to an inhalation inlet port 9 projecting from the front face 5 of the wall at its lower end. A short length of a corrugated flexible tube 10 with a mouthpiece 11 at one end removably connects at its opposite end to the inhalation port 9.

Inside the housing 1 the cylinder 4 contains a lightweight piston 20 that is a close sliding and sealing fit within the cylinder. The curved, outer surface of the piston 20 is conspicuously marked or coloured so that it is clearly visible through the wall of the cylinder 4. In its natural state the mass of the piston 20 and the force of gravity cause it to sit at the bottom of the cylinder. The cylinder 4 has graduation marks 14 along its length from the upper end of the piston 20 in its rest position to the upper end of the cylinder, typically these graduations extend from 0 mL at the lower end to 4000 mL at the upper end to indicate the volume of air inhaled from the cylinder. The apparatus also includes a manually-movable slider 21 that is movable up or down the left-hand side of the cylinder 4 and is held in place by friction. The slider 21 is manually positioned by the user along the cylinder 4 before use to the desired target volume to be achieved.

The apparatus also includes an electronic pressure sensor 30 located inside the housing 1 in a position where it will be exposed to changes in air flow or pressure within the housing caused by use. The sensor 30 is shown positioned in the gas passage 8 but it could be positioned in the upper end of the cylinder 4, at the inlet port 9 or it could be positioned in the apparatus outside the housing 1 such as in the tube 10 or mouthpiece 11. Typically, the sensor 30 is a differential pressure sensor responsive to pressure difference across it within the passage 8. The sensor 30 is arranged to provide a wireless output signal, such as by radio frequency transmission at Bluetooth protocol. The signal is received by a monitor 40 including a memory 41 and processor 42 where the pressure signal is processed and stored, and a screen 43 on which feedback is provided to the user. The sensor could be an RFID sensor that is powered when interrogated by an external RFID reader. The monitor could be provided by a dedicated unit or by a program or app in a smart phone, tablet or the like. Alternatively, the sensor could be connected by a cable to a monitor mounted on the outside of the spirometer housing.

Instead of a pressure sensor it would be possible to use a sensor responsive to flow. Such a flow sensor could include a turbine driven by flow, a hot-wire anemometer, a vibrating piezo element such as of the kind described in WO14108658 or any other conventional flow-sensing device.

In use, the user connects the tube 10 to the port 9, turns on the monitor 40 and positions the slider 21 to the target volume. The user then exhales completely, puts the mouthpiece 11 to his mouth and inhales deeply and slowly. This causes pressure in the cylinder 4 above the piston 20 to drop and also causes a pressure differential at the sensor 30 in the passage 8, thereby changing the output from the sensor to the monitor 40. The result of this is that the piston 20 moves up the cylinder 4 towards the target volume indicated by the slider 21. As the piston 20 rises up the cylinder 4 air is drawn into the cylinder beneath the piston via the vent aperture 6. The user then attempts to hold the piston 20 at the target volume for as long as possible. He then exhales, allowing the piston 20 to fall under gravity, forcing air beneath the piston out of the cylinder 4 via the vent 6. The user then repeats the inhalation breath for a prescribed number of times after suitable rest pauses.

The monitor 40 is arranged to process the output signals from the sensor 30 and utilise these to provide immediate feedback to the user regarding use of the apparatus. The monitor 40 also maintains a record of the user's progress over a given period, such as over one or two months. The immediate feedback could be provided by a visual cue, such as a change of colour or a change of frequency of a pulsing image on the screen 43, or by an audible cue, such as by generating a sound when the target volume is reached and generating a different sound when the target volume has been maintained for a target time. Feedback could instead be provided in other ways, such as by a tactile cue produced by vibration. The longer term record of the user's progress could be provided by a graphical representation showing the change in performance over a particular period.

Instead of having a vertical cylinder and using gravity to provide the force restoring the piston to one end of the cylinder it would be possible to orient the cylinder away from the vertical and use something else to provide the restoring force, such as a spring.

The arrangement of the present invention enables useful feedback to be provided to the user to encourage use of the spirometer even when the user only achieves below the target levels. The arrangement also avoids the need to obscure the piston so that this can be clearly seen by the user.

1-7. (canceled)

8. Incentive spirometer apparatus having a housing, a cylinder, a patient inhalation inlet and a gas passage between the inhalation inlet and one end of the cylinder, the cylinder containing a piston movable along the cylinder against a restoring force, such that an inhalation breath applied to the inlet causes a reduced pressure at one end of the cylinder so as to displace the piston against the restoring force, characterised in that the spirometer apparatus also includes an electronic pressure or flow sensor responsive to change in pressure or flow of gas within the apparatus during use and a monitor for receiving an output signal from the sensor and for processing and utilising the output signal.

9. Apparatus according to claim 8, characterised in that the sensor is arranged to provide a wireless output to the monitor and that the monitor is located externally of the spirometer housing.

10. Apparatus according to claim 8, characterised in that the housing is arranged to stand with the cylinder extending vertically and that the restoring force is provided by the effect of gravity on the mass of the piston.

11. Apparatus according to claim 8, characterised in that the apparatus includes a flexible tube having one end connected with the inhalation inlet and with a mouthpiece at its opposite end.

12. Apparatus according to claim 8, characterised in that the sensor is located in the gas passage between the inhalation inlet and the one end of the cylinder.

13. Apparatus according to claim 8, characterised in that the monitor is provided by a mobile phone arranged to receive wireless signals from the sensor, and that the mobile phone is programmed to provide an indication to the user of feedback regarding use of the apparatus.

14. Apparatus according to claim 8, characterised in that the apparatus includes a slider that can be manually positioned by the user along the cylinder to indicate a target volume.
